(19) United States
(12) Patent Application Publication
(10) Pub. No.: US 2024/0065711 A1
(43) Pub. Date: Feb. 29, 2024

(54) LITHOTRIPSY CATHETERS

(71) Applicant: BOSTON SCIENTIFIC SCIMED, INC., MAPLE GROVE, MN (US)

(72) Inventor: Ryan Hendrickson, Albertville, MN (US)

(73) Assignee: BOSTON SCIENTIFIC SCIMED, INC., MAPLE GROVE, MN (US)

(21) Appl. No.: 18/236,596

(22) Filed: Aug. 22, 2023

Related U.S. Application Data
(60) Provisional application No. 63/400,167, filed on Aug. 23, 2022.

(51) Int. Cl. A61B 17/22

(52) U.S. Cl. CPC A61B 17/2202; A61B 2017/22051; A61M 25/0074

(57) ABSTRACT

Methods and systems for breaking down an intravascular lesion. An illustrative method may comprise advancing a catheter through the vasculature to a target location. The catheter may a catheter shaft, an expandable member secured to a distal portion of the catheter shaft, and one or more ultrasound transducers. The method may further comprise activating the ultrasound transducer to emit an ultrasound field directed towards the target location.

### CROSS REFERENCE TO RELATED APPLICATIONS

The present application claims the benefit of and priority to US Provisional Patent Application No. 63/400,167, filed Aug. 23, 2022, the disclosure of which is incorporated herein by reference.

### TECHNICAL FIELD

The disclosure pertains to catheters with an internal lithotripsy emitter. More particularly, the disclosure is directed to angioplasty devices for modifying lesion compliance.

### BACKGROUND

Many patients suffer from occluded arteries and other blood vessels which restrict blood flow. Occlusions can be partial occlusions that reduce blood flow through the occluded portion of a blood vessel or total occlusions (e.g., chronic total occlusions) that substantially block blood flow through the occluded blood vessel. Occluded, stenotic, or narrowed blood vessels may be treated with a number of relatively non-invasive medical procedures including percutaneous transluminal angioplasty (PTA), percutaneous transluminal coronary angioplasty (PTCA), atherectomy, and lithotripsy. However, the efficacy of intravascular lithotripsy may be reduced for eccentric calcific lesions. Of the known medical devices, systems, and methods, each has certain advantages and disadvantages. There is an ongoing need to provide alternative medical devices and systems, including devices and systems for treating occlusions or calcified lesions.

### SUMMARY

The disclosure is directed to several alternative designs, materials and methods of manufacturing medical device structures and assemblies.

In a first example a method for breaking down an intravascular lesion may comprise advancing a catheter through a vasculature system to a target location and activating the one or more ultrasound transducers to emit an ultrasound field, the ultrasound field directed towards the target location. The catheter may comprise a catheter shaft, an expandable member secured to a distal portion of the catheter shaft, and one or more ultrasound transducers.

Alternatively or additionally to any of the examples above, in another example, the catheter may further comprise a guidewire extending through a lumen of the catheter shaft.

Alternatively or additionally to any of the examples above, in another example, at least one of the one or more ultrasound transducers may be coupled to the expandable member.

Alternatively or additionally to any of the examples above, in another example, the catheter shaft may comprise an outer tubular member and an inner tubular member.

Alternatively or additionally to any of the examples above, in another example, at least one of the one or more ultrasound transducers may be coupled to the inner tubular member.

Alternatively or additionally to any of the examples above, in another example, the expandable member may comprise an inflatable balloon.

Alternatively or additionally to any of the examples above, in another example, the expandable member may comprise an expandable basket.

Alternatively or additionally to any of the examples above, in another example, the expandable basket may comprise two or more lobes.

Alternatively or additionally to any of the examples above, in another example, the expandable basket may further comprise one or more slide rings positioned between and coupled to the two or more lobes.

Alternatively or additionally to any of the examples above, in another example, the expandable basket may comprise one or more longitudinally extending struts.

Alternatively or additionally to any of the examples above, in another example, the expandable basket may comprise a helically extending strut.

Alternatively or additionally to any of the examples above, in another example, the expandable basket may comprise a woven structure.

Alternatively or additionally to any of the examples above, in another example, the one or more ultrasound transducers may be arranged in one or more arrays.

Alternatively or additionally to any of the examples above, in another example, the ultrasound field may be emitted radially from the catheter.

Alternatively or additionally to any of the examples above, in another example, the ultrasound field may be emitted in a direction parallel to a longitudinal axis of the catheter shaft.

In another example, a method for breaking down an intravascular lesion may comprise advancing a catheter through a vasculature system to a target location and activating the one or more ultrasound transducers to emit an ultrasound field, the ultrasound field directed towards the target location. The catheter may comprise a catheter shaft comprising an outer tubular member and an inner tubular member, an inflatable balloon secured to a distal portion of the catheter shaft; and one or more ultrasound transducers.

Alternatively or additionally to any of the examples above, in another example, at least one of the one or more ultrasound transducers may be coupled to the inflatable balloon.

Alternatively or additionally to any of the examples above, in another example, at least one of the one or more ultrasound transducers may be coupled to the inner tubular member.

Alternatively or additionally to any of the examples above, in another example, the ultrasound field may be emitted radially from the catheter.

Alternatively or additionally to any of the examples above, in another example, the ultrasound field may be emitted in a direction parallel to a longitudinal axis of the catheter shaft.

In another example, method for breaking down an intravascular lesion may comprise advancing a catheter through a vasculature system to a target location and activating the one or more ultrasound transducers to emit an ultrasound field, the ultrasound field directed towards the target location. The catheter may comprise a catheter shaft comprising an outer tubular member and an inner tubular member, an expandable basket secured to a distal portion of the catheter shaft, and one or more ultrasound transducers.

Alternatively or additionally to any of the examples above, in another example, at least one of the one or more ultrasound transducers may be coupled to the expandable basket.

Alternatively or additionally to any of the examples above, in another example, at least one of the one or more ultrasound transducers may be coupled to the inner tubular member.

Alternatively or additionally to any of the examples above, in another example, the expandable basket may comprise two or more lobes.

Alternatively or additionally to any of the examples above, in another example, the expandable basket may comprise one or more longitudinally extending struts.

Alternatively or additionally to any of the examples above, in another example, the expandable basket may comprise a helically extending strut.

Alternatively or additionally to any of the examples above, in another example, the expandable basket may comprise a woven structure.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a side view of an illustrative lithotripsy balloon catheter disposed in a blood vessel;

FIG. 2 is a side view of another illustrative lithotripsy balloon catheter;

FIG. 3 is a side view of an illustrative lithotripsy catheter disposed in a blood vessel;

FIG. 4 is a side view of an illustrative lithotripsy guidewire disposed in a blood vessel;

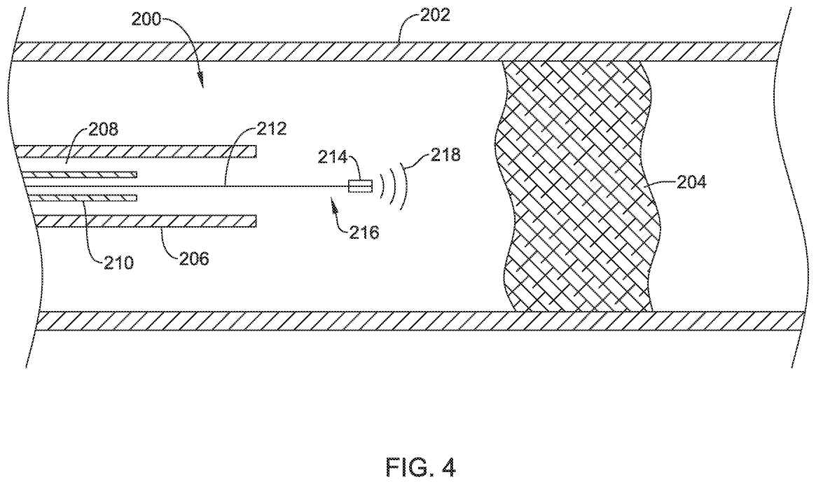

FIG. 5 is a side view of an illustrative expandable frame lithotripsy catheter disposed in a blood vessel;

FIG. 6 is a side view of another illustrative expandable frame lithotripsy catheter disposed in a blood vessel;

FIG. 7 is a side view of another illustrative expandable frame lithotripsy catheter disposed in a blood vessel;

FIG. 8 is a side view of another illustrative expandable frame lithotripsy catheter disposed in a blood vessel; and

FIG. 9 is a side view of another illustrative expandable frame lithotripsy catheter disposed in a blood vessel.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Many patients suffer from occluded arteries, other blood vessels, and/or occluded ducts or other body lumens which may restrict bodily fluid (e.g., blood, bile, etc.) flow. Occlusions can be partial occlusions that reduce blood flow through the occluded portion of a blood vessel or total occlusions (e.g., chronic total occlusions) that substantially block blood flow through the occluded blood vessel. Revascularization techniques include using a variety of devices to pass through the occlusion to create or enlarge an opening through the occlusion. In some cases, lesions such as calcified lesions may create problems for revascularization techniques, and it may be beneficial to treat the calcified lesions in order to modify their compliance to enable full dilation before stenting.

In some cases, for example, ultrasound may be used to treat vascular lesions, such as fibrotic and calcified lesions, at various states of disease progression, ranging from soft plaques to severely calcified lesions. Vascular lesions that may lend themselves to being treated with ultrasound-based devices include irregular, severely calcified plaques that are located within and adjacent to vessel walls, and lesions that are more or less rigid and thus may be susceptible to being mechanically fatigued to failure. For example, sound-based devices may be used to produce standing wave pressure patterns within the thickness of the lesion, bending moments at the ends of the lesion, and/or resonance along the length of the lesion. In some cases, the high frequency mechanical action of ultrasound may also be effective in treating earlier state vascular lesions, including fibrotic and soft plaques. In some cases, an ultrasound device may apply a treatment of unfocused, near-field ultrasound waves to treat vascular lesions. While the devices or systems described herein are described with respect to vascular lesions, it should be understood that the devices or systems may be used in other applications, such as, but not limited to, peripheral calcified lesions, aortic valves, mitral valves, or non-vascular applications including the treatment of tumors. For example, the methods and systems described herein may be used in any conduit that includes or is adjacent to a target treatment site such as, but not limited to vascular lesions, peripheral lesions, tumors, etc.

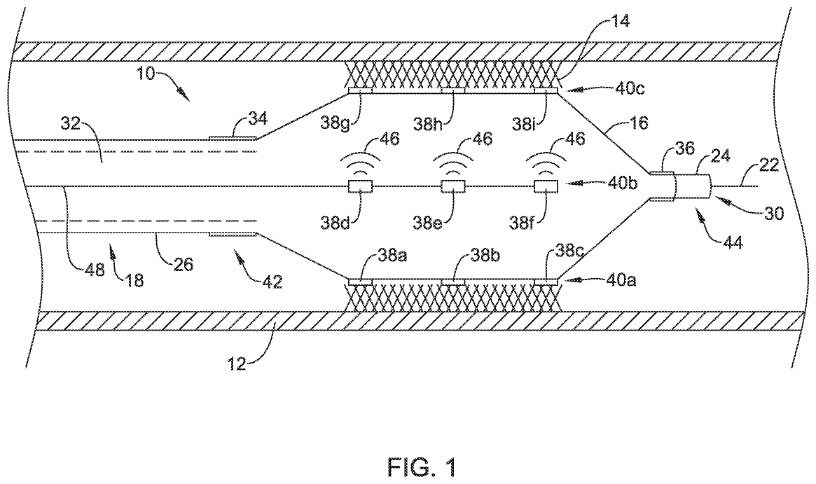

FIG. 1 is a side view of an illustrative catheter 10 that may be used to treat a lesion disposed in a blood vessel 12 and positioned adjacent to an intravascular lesion 14. The catheter 10 may be configured to emit a shockwave or ultrasound field. The catheter 10 may include a balloon 16 coupled to a catheter shaft 18. In general, the catheter 10 may be advanced over a guidewire 22, through the vasculature, to a target area with the balloon 16 in a collapsed or deflated configuration. Once positioned at the target location in the vasculature, the balloon 16 can be inflated to exert a radially outward force on the lesion 14. The target area may be within any suitable peripheral or cardiac vessel lumen location.

The balloon 16 may be made from typical angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. The balloon 16 may be compliant, semi-compliant, or non-compliant, as desired. In some embodiments, the balloon 16 may be a high-pressure balloon.

The shaft 18 may be a catheter shaft, similar to typical catheter shafts. For example, the catheter shaft 18 may include an outer tubular member 26 and an inner tubular member 24 extending through at least a portion of the outer tubular member 26. The inner and outer tubular members 24, 26 may be manufactured from a number of different materials. For example, the inner and outer tubular members 24, 26 may be made of metals, metal alloys, polymers, metal-polymer composites or any other suitable materials.

The outer tubular member 26 may extend proximally from a distal end region 42 to the proximal end configured to remain outside of a patient's body. The inner tubular member 24 may extend proximally from a distal end region 44 to a proximal end configured to remain outside of a patient's body. Although not shown, the proximal ends of the inner and/or outer tubular members 24, 26 may include a hub attached thereto for connecting other treatment devices or providing a port for facilitating other treatments. It is contemplated that the stiffness of the inner and/or outer tubular members 24, 26 may be modified to form a catheter 10 for use in various vessel diameters and various locations within the vascular tree.

The inner and outer tubular members 24, 26 may be arranged in any appropriate way. For example, in some embodiments, the inner tubular member 24 can be disposed coaxially within the outer tubular member 26. According to these embodiments, the inner and outer tubular members 24, 26 may or may not be secured to one another along the general longitudinal axis of the catheter shaft 18. Alternatively, the inner tubular member 24 may follow the inner wall or otherwise be disposed adjacent the inner wall of the outer tubular member 26. In other embodiments, the inner and outer tubular members 24, 26 may be arranged in another desired fashion. In some embodiments, the inner tubular member 24 and/or outer tubular member 26 may be torqueable to facilitate rotation of the device 10. The inner tubular member 24 and/or outer tubular member 26 may include an embedded reinforcing member, such as, but not limited to, an embedded coil or braided member.

The inner tubular member 24 may include an inner lumen 30. In at least some embodiments, the inner lumen 30 is a guidewire lumen for receiving the guidewire 22 therethrough. Accordingly, the catheter 10 can be advanced over the guidewire 22 to the desired location. The guidewire lumen 30 may extend along essentially the entire length of the catheter shaft 18 such that catheter 10 resembles traditional “over-the-wire” catheters. Alternatively, the guidewire lumen 30 may extend along only a portion of the catheter shaft 18 such that the catheter 10 resembles “single-operator-exchange” or “rapid-exchange” catheters.

The catheter shaft 18 may also include an inflation lumen 32 that may be used, for example, to transport inflation media to and from the balloon 16 to selectively inflate and/or deflate the balloon 16. The location and position of the inflation lumen 32 may vary, depending on the configuration of the inner and outer tubular members 24, 26. For example, when the outer tubular member 26 surrounds the inner tubular member 24, the inflation lumen 32 may be defined within the space between the outer tubular member 26 and the inner tubular member 24. In embodiments in which the outer tubular member 26 is disposed alongside the inner tubular member 24, then the inflation lumen 32 may be the lumen of the outer tubular member 26.

The balloon 16 may be coupled to the catheter shaft 18 in any of a number of suitable ways. For example, the balloon 16 may be adhesively or thermally bonded to the catheter shaft 18. In some embodiments, a proximal waist 34 of the balloon 16 may be bonded to the catheter shaft 18, for example, bonded to the distal end region 42 of the outer tubular member 26, and a distal waist 36 of the balloon 16 may be bonded to the catheter shaft 18, for example, bonded to the distal end region 44 of the inner tubular member 24. The exact bonding positions, however, may vary. It is further contemplated that the balloon 16 may take other shapes, as desired. For example, the balloon 16 may include more than one chamber or lobe.

One or more ultrasound transducers or emitters 38a-i (collectively, 38) may be coupled to the balloon 16. The ultrasound transducers 38 may be glued, bonded, adhered, etc. to the balloon 16. The balloon 16 may include any number of ultrasound transducers 38 desired, such as, but not limited to, one, two, three, four, five, ten, twenty, or more, etc. The one or more ultrasound transducers 38 may be coupled to an outer surface of the balloon 16. Alternatively, or additionally, the one or more ultrasound transducers 38 may be coupled to an inner surface of the balloon 16. It is contemplated that locating the ultrasound transducers 38 on the balloon 16 may bring the ultrasound transducers in closer proximity to the lesion 14 for a targeted and/or localized therapy. The ultrasound waves 46 may be transmitted through the vessel wall 12. In some cases, the one or more ultrasound transducers 38 may include a piezoelectric material, which transmits acoustic pressure in response to an applied voltage. The one or more ultrasound transducers 38 may be driven at one or more frequencies in the range of about 20 kilohertz (kHz) to about 50 megahertz (MHz). The one or more ultrasound transducers 38 may be a single ultrasound transducer, or the one or more ultrasound transducers 38 may include a series of ultrasound transducers that may be operated to effectively function as a single ultrasound transducer, providing the desired acoustic pressure over the desired treatment area. It is further contemplated that each ultrasound transducer 38 may be operated independently from other ultrasound transducers 38. The acoustic pressure applied may range from tens of kiloPascals (kPa) to in excess of ten megaPascals (MPa).

The one or more ultrasound transducers 38 may be arranged in any configuration desired. In some examples, the ultrasound transducers 38 may be staggered about the circumference and/or length of the balloon 16 such that a maximum number of ultrasound transducers 38 can be positioned on the balloon 16. In another example, as shown in FIG. 1, the one or more ultrasound transducers 38 may be arranged in linear arrays 40a, 40b, 40c (collectively, 40) with each array 40 extending generally parallel to a longitudinal axis of the catheter shaft 18. For example, a first plurality of ultrasound transducers 38a, 38b, 38c may form a first array 40a, a second plurality of ultrasound transducers 38d, 38e, 38f may form a second array 40b, and a third plurality of ultrasound transducers 38g, 38h, 38i may form a third array 40c. The arrays 40 may be uniformly or eccentrically spaced about a circumference of the balloon 16. In the illustrated embodiment of FIG. 1, each array 40 is spaced about 90° from adjacent arrays 40. However, this is not required. the spacing may be determined, in part, by the number of arrays 40 on the balloon 16. Further, the arrays need not be uniformly spaced. In some examples, the one or more ultrasound transducers 38 may be provided as circumferentially extending arrays with each array extending about a circumference of the balloon 16. In yet other embodiments, the one or more ultrasound transducers 38 may be arranged in a helical manner along the balloon 16. For example, an ultrasound transducer 38 may be longitudinally and circumferentially spaced from each adjacent ultrasound transducer 38. These are just some examples. It can be appreciated that the one or more ultrasound transducers 38 may be positioned in any arrangement, as desired. In some embodiments, each array 40 may function independently from other arrays or the arrays 40 may collectively be operated to effectively function as a single ultrasound transducer.

Each of the one or more ultrasound transducers 38 may produce an ultrasound field 46 that includes a near field region and a far field region. In the near field region, dynamic acoustic pressures may be cyclically applied to the calcified lesion 14. As used herein, the near field region refers to a region in close proximity radially to a surface of the ultrasound transducer 38, for example, the region extending outward from the transducer surface to a radial distance less than or equal to a length of the ultrasound transducer 38, wherein the acoustic pressure waves transmitted by the ultrasound transducer 38 are unfocused and can be controlled to be substantially uniform upon the calcified lesion 14. The ultrasound transducers 38 may be configured to emit ultrasound waves in two opposing directions, or about 180° apart. In other examples, the ultrasound transducers 38 may be configured to emit ultrasound waves in a single direction. For example, the ultrasound sound transducers 38 may be configured to emit ultrasound waves in a direction radially outward from the balloon 16. When two or more ultrasound transducers 38 are provided, the ultrasound transducers 38 may be arranged to emit ultrasound waves in differing radial and/or axial directions, although this is not required.

In some cases, for example, the ultrasound transducer 38 may be configured to impart a uniform or substantially uniform acoustic pressure along the length of the calcified lesion 14. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter. Depending on the therapeutic applications, the ultrasound transducer 38 may be configured to impart a uniform or substantially uniform acoustic pressure over a length of about 10 mm to about 60 mm at a radial distance of about 1 mm to about 8 mm as measured from a central axis extending through the catheter shaft 18. Multiple ultrasound transducers 38 may be used and configured to extend the effective therapeutic length, such as up to a length of about 200 mm.

To impart a uniform or substantially uniform acoustic pressure in the near field, the ultrasound transducers 38 may have a length that is multiple times larger than a diameter of the inner tubular member 24 and/or the outer tubular member 26. In some cases, the ultrasound transducers 38 may have a length that is at least as long as a length of the calcified lesion 14, to generate a uniform or substantially uniform acoustic pressure over a length of about 20 to about 80 mm. As described above, the ultrasound transducers 38, may be a single ultrasound transducer or a series of ultrasound transducers or transducer elements driven in such a way as to effectively act as a single ultrasound transducer. The ultrasound transducers 38 may be electrically coupled to an electronic source (not explicitly shown) via one or more wires 48. In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 14.

The balloon 16 may be inflated using any suitable inflation fluid. Example inflation fluid may include, but is not limited to, water, saline (e.g., 0.9% sodium chloride), a mixture of saline and a radiopaque contrast agent (e.g., a 50/50 mixture), etc. In some cases, the inflation fluid may be chosen for how acoustic energy transmits through the inflation fluid. It will be appreciated that by selecting a particular fluid with which to inflate the balloon 16, one is able to control the efficiency of acoustic energy transmission through the fluid and to the calcified lesion 14. In one example, the inflation fluid may be chosen to have a specific characteristic acoustic impedance to serve as an acoustic matching between the ultrasound transducers 38 and the vessel wall 12. In another example, the inflation fluid may be chosen to have a specific characteristic acoustic impedance to serve as an acoustic matching to minimize transmission loss across a wall of the inflatable balloon 16. In another example, the inflation fluid may be chosen to have a specific sound velocity in order to modify the near field behavior of the ultrasound transducers 38.

In use, the catheter 10 may be advanced through the vasculature to a desired treatment region. Once the catheter 10 is at the treatment region, the balloon 16 may then be expanded. Energy may then be supplied to the ultrasound transducers 38. The expansion of the balloon 16 and/or the application of the sound energy may break up the lesion 14 to dilate or expand the vessel 12. While not explicitly shown, the ultrasound transducers 38 may be connected to a single control unit or to separate control units by one or more electrical conductors 48. The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 10 may be longitudinally repositioned and energy may once again be delivered to the ultrasound transducers 38. If necessary, the catheter 10 may be rotated to break up the lesion 14 around the circumference of the vessel at each longitudinal location. This process may be repeated at any number of longitudinal locations desired. It is contemplated that the balloon 16 may be deflated or partially deflated to allow for movement of the catheter 10.

When the procedure has been completed, the balloon 16 may be deflated or collapsed for withdrawal from the body. It is contemplated that balloon 16 and ultrasound transducers 38 may collapse in such a manner that all or part of the ultrasound transducers 38 and the associated electronics are encased within folds of the deflated balloon to reduce potential “catch” points on the catheter 10 which may also reduce the force required to withdraw the catheter 10.

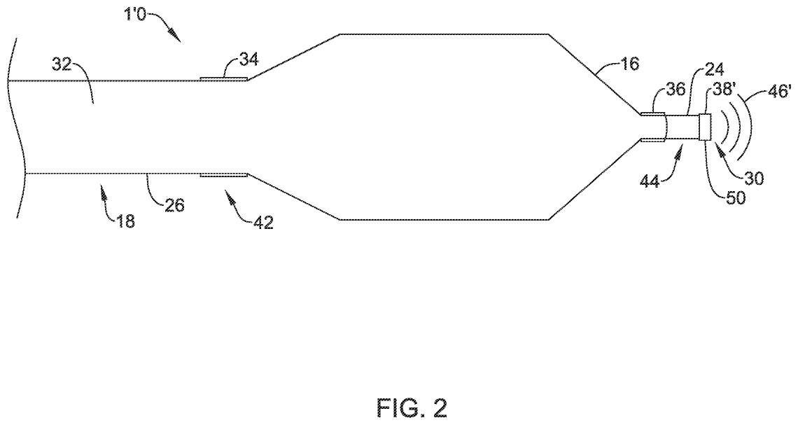

It is contemplated that the ultrasound transducers 38 may be positioned at locations in addition to or in place of the balloon 16. FIG. 2 illustrates a side view of another illustrative catheter 10′ with an ultrasound transducer 38′ positioned adjacent to the distal end region 44 of the inner tubular member 24. The catheter 10′ may be similar in form and function to the catheter 10 described with respect to FIG. 1 where the same or similar reference numbers will be used to refer to the same or like parts. The ultrasound transducer 38′ may be positioned at or proximate to a distal end 50 of the catheter 10′ and may be configured to direct the ultrasound field 46′ in a direction that is generally parallel to or in line with a longitudinal axis of the catheter shaft 18. For example, the ultrasound field 46′ may be configured to break up chronic total occlusions. In instances where the plaque/lesion precludes the passages of the catheter 10′, the ultrasound transducer 38′ may be activated to break down the plaque/lesion and allow for the passage of the catheter 10′. While only a single transducer 38′ is illustrated, it is contemplated that the catheter 10′ may include more than one ultrasound transducer 38′. It is further contemplated that additional ultrasound transducers 38′ may be provided on the balloon 16, as shown in FIG. 1. When more than one ultrasound transducer 38′ is provided, the ultrasound transducers 38′ may be configured to be individually or collectively activated, as desired. While not explicitly shown, an electrical connector may extend from the ultrasound transducer 38′ to a control unit configured to remain outside the body. The electrical conductor may be disposed within a lumen 30, 32 of the catheter shaft 18 or along an exterior of the catheter 10′, as desired.

In use, the catheter 10′ may be advanced through the vasculature to a desired treatment region. Once the catheter 10′ is at the treatment region, the balloon 16 may then be expanded to anchor and/or center the catheter 10′, although this is not required. Energy may then be supplied to the ultrasound transducer 38′. The application of the sound energy may break up the plaque/lesion to allow the catheter 10′ to pass the occlusion. In some cases, once the occlusion has been broken up, the catheter 10′ may be longitudinally advanced and the balloon 16 expanded at the lesion to dilate or expand the vessel. While not explicitly shown, the ultrasound transducers 38 may be connected to a single control unit or to separate control units by one or more electrical conductors. The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 10′ may be longitudinally repositioned and energy may once again be delivered to the ultrasound transducer 38′. If necessary, the catheter 10′ may be rotated to break up the lesion around the circumference of the vessel at each longitudinal location. This process may be repeated at any number of longitudinal locations desired. It is contemplated that the balloon 16 may be deflated or partially deflated to allow for movement of the catheter 10′. When the procedure has been completed, the balloon 16 may be deflated or collapsed for withdrawal from the body.

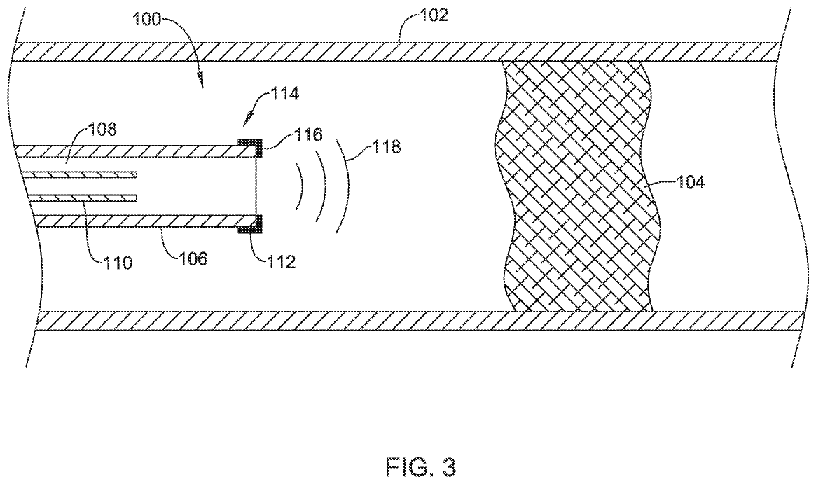

FIG. 3 is a cross-sectional view of another illustrative catheter system 100 that may be used to break up plaque and/or lesions. The figure depicts the system 100 within a blood vessel 102 of a patient having a total occlusion 104. The system 100 may include a guide catheter 106 having a lumen 108 extending therethrough. Another medical device 110 may extend through the lumen 108 of the guide catheter 106. The “second” medical device 110 may be, for example, a stent delivery system, an angioplasty catheter, a dilation catheter, a cutting balloon catheter, a rotational atherectomy catheter, or the like. During use, the medical device 110 may be advanced through the guide catheter 106 and over a guidewire (not explicitly shown) to a position adjacent to an area of interest. When properly positioned, the medical device 110 may be used to perform a suitable diagnostic and/or treatment intervention.

One or more ultrasound transducers 112 may be coupled adjacent a distal end region 114 of the guide catheter 106. In some embodiments, the one or more ultrasound transducers 112 may be affixed to an outer surface of the guide catheter 106. In such an instance, the one or more ultrasound transducers 112 may extend about an entire perimeter of the guide catheter 106 or less than an entire perimeter of the guide catheter, as desired. Alternatively, or additionally, the one or more ultrasound transducers 112 may be coupled to the distal end surface 116 of the guide catheter 106 which extends generally orthogonal to a longitudinal axis of the guide catheter.

The guide catheter 106 may include any number of ultrasound transducers 112 desired, such as, but not limited to, one, two, three, four, five, ten, twenty, or more, etc. In some cases, the one or more ultrasound transducers 112 may include a piezoelectric material, which transmits acoustic pressure in response to an applied voltage. The one or more ultrasound transducers 112 may be driven at one or more frequencies in the range of about 20 kilohertz (kHz) to about 50 megahertz (MHz). The one or more ultrasound transducers 112 may be a single ultrasound transducer, or the one or more ultrasound transducers 112 may include a series of ultrasound transducers that may be operated to effectively function as a single ultrasound transducer, providing the desired acoustic pressure over the desired treatment area. It is further contemplated that each ultrasound transducer 112 may be operated independently from other ultrasound transducers 112. The acoustic pressure applied may range from tens of kiloPascals (kPa) to in excess of ten megaPascals (MPa).

The one or more ultrasound transducers 112 may be arranged in any configuration desired. In some examples, the ultrasound transducers 112 may be staggered about the circumference and/or length of the guide catheter 106 such that a maximum number of ultrasound transducers 112 can be positioned on the guide catheter 106. In another example, the one or more ultrasound transducers 112 may be arranged in linear arrays with each array extending generally parallel to a longitudinal axis of the guide catheter 106. When so provided, the arrays may be uniformly or eccentrically spaced about a circumference of the guide catheter 106. In some examples, the one or more ultrasound transducers 112 may be provided as circumferentially extending arrays with each array extending about a circumference of the guide catheter 106. In yet other embodiments, the one or more ultrasound transducers 112 may be arranged in a helical manner along the guide catheter 106. For example, an ultrasound transducer 112 may be longitudinally and circumferentially spaced from each adjacent ultrasound transducer 112. These are just some examples. It can be appreciated that the one or more ultrasound transducers 112 may be positioned in any arrangement, as desired. In some embodiments, each ultrasound transducer 112 may function independently from other transducers or the ultrasound transducers 112 may be collectively operated to effectively function as a single ultrasound transducer.

Each of the one or more ultrasound transducers 112 may produce an ultrasound field 118 that includes a near field region and a far field region. In the near field region, dynamic acoustic pressures may be cyclically applied to the calcified lesion 104. As used herein, the near field region refers to a region in close proximity radially to a surface of the ultrasound transducer 112, for example, the region extending outward from the transducer surface to a radial distance less than or equal to a length of the ultrasound transducer 112, wherein the acoustic pressure waves transmitted by the ultrasound transducer 112 are unfocused and can be controlled to be substantially uniform upon the calcified lesion 104. The ultrasound transducers 112 may be configured to emit ultrasound waves in two opposing directions, or about 180° apart. In other examples, the ultrasound transducers 112 may be configured to emit ultrasound waves in a single direction. For example, the ultrasound sound transducers 112 may be configured to emit ultrasound waves in a direction radially outward from the guide catheter 106 or in a direction generally parallel to the longitudinal axis of the guide catheter 106. When two or more ultrasound transducers 112 are provided, the ultrasound transducers 112 may be arranged to emit ultrasound waves in differing radial and/or axial directions, although this is not required.

In some cases, for example, the ultrasound transducer 112 may be configured to impart a uniform or substantially uniform acoustic pressure to an end of the calcified lesion 104. As the lesion 104 breaks down, the guide catheter 106 may be advanced to break up a length of the lesion 104. In some instances, ultrasound transducers 112 may be positioned to emit ultrasound waves in a direction generally parallel to the longitudinal axis of the guide catheter 106 to create a path through the lesion 104 for the guide catheter 106. Additional ultrasound transducers 112 may be positioned along a length of the guide catheter 106 to emit radially extending ultrasound waves to further break down the lesion 104 as the guide catheter 106 is passed therethrough. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter.

Depending on the therapeutic applications, the ultrasound transducer 112 may be configured to impart a uniform or substantially uniform acoustic pressure over a length of about 10 mm to about 60 mm at a radial distance of about 1 mm to about 8 mm as measured from a central axis extending through the catheter shaft 18. Multiple ultrasound transducers 112 may be used and configured to extend the effective therapeutic length, such as up to a length of about 200 mm.

To impart a uniform or substantially uniform acoustic pressure in the near field, the ultrasound transducers 112 may have a length that is multiple times larger than a diameter of the guide catheter 106. In some cases, the ultrasound transducers 112 may have a length that is at least as long as a length of the calcified lesion 104, to generate a uniform or substantially uniform acoustic pressure over a length of about 20 to about 80 mm. As described above, the ultrasound transducers 112, may be a single ultrasound transducer or a series of ultrasound transducers or transducer elements driven in such a way as to effectively act as a single ultrasound transducer. The ultrasound transducers 112 may be electrically coupled to an electronic source (not explicitly shown) via one or more wires (not explicitly shown). In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 104.

In use, the guide catheter 106 may be advanced through the vasculature to a desired treatment region. Energy may then be supplied to the ultrasound transducer 112. The application of the sound energy may break up the plaque/lesion to allow the guide catheter 106 to pass the occlusion 104. In some cases, once the occlusion 104 has been broken up, the guide catheter 106 may be longitudinally advanced. While not explicitly shown, the ultrasound transducers 112 may be connected to a single control unit or to separate control units by one or more electrical conductors. The amount of energy delivered to the ultrasound transducers may be determined by the desired treatment as well as the feedback provided by other components of the catheter or other devices.

Once a particular location has been treated, it may be desirable to perform further procedures at different longitudinal locations. The guide catheter 106 may be longitudinally repositioned and energy may once again be delivered to the ultrasound transducer 112. If necessary, the guide catheter 106 may be rotated to break up the lesion around the circumference of the vessel at each longitudinal location. This process may be repeated at any number of longitudinal locations desired.

FIG. 3 is a cross-sectional view of another illustrative catheter system 200 that may be used to break up plaque and/or lesions. The figure depicts the system 200 within a blood vessel 202 of a patient having a total occlusion 204. The system 200 may include a guide catheter 206 having a lumen 208 extending therethrough. Another medical device 210 may extend through the lumen 208 of the guide catheter 206. The “second” medical device 210 may be, for example, a stent delivery system, an angioplasty catheter, a dilation catheter, a cutting balloon catheter, a rotational atherectomy catheter, or the like. During use, the medical device 210 may be advanced through the guide catheter 206 and over a guidewire 212 to a position adjacent to an area of interest. When properly positioned, the medical device 210 may be used to perform a suitable diagnostic and/or treatment intervention. One or more ultrasound transducers 214 may be coupled adjacent a distal end region 216 of the guidewire 212. In some embodiments, the one or more ultrasound transducers 214 may be affixed to an outer surface of the guidewire 212. In such an instance, the one or more ultrasound transducers 214 may extend about an entire perimeter of the guidewire 212 or less than an entire perimeter of the guidewire 212, as desired. Alternatively, or additionally, the one or more ultrasound transducers 214 may be coupled to the distal end surface of the guidewire 212 which extends generally orthogonal to a longitudinal axis of the guidewire 212. While not explicitly shown, ultrasound transducers 214 may be additionally provided on the guide catheter 206, as described with respect to FIG. 3.

The guidewire 212 may include any number of ultrasound transducers 214 desired, such as, but not limited to, one, two, three, four, five, ten, twenty, or more, etc. In some cases, the one or more ultrasound transducers 214 may include a piezoelectric material, which transmits acoustic pressure in response to an applied voltage. The one or more ultrasound transducers 214 may be driven at one or more frequencies in the range of about kilohertz (kHz) to about 50 megahertz (MHz). The one or more ultrasound transducers 214 may be a single ultrasound transducer, or the one or more ultrasound transducers 214 may include a series of ultrasound transducers that may be operated to effectively function as a single ultrasound transducer, providing the desired acoustic pressure over the desired treatment area. It is further contemplated that each ultrasound transducer 214 may be operated independently from other ultrasound transducers 214. The acoustic pressure applied may range from tens of kiloPascals (kPa) to in excess of ten megaPascals (MPa).

The one or more ultrasound transducers 214 may be arranged in any configuration desired. In some examples, the ultrasound transducers 214 may be staggered about the circumference and/or length of the guidewire 212 such that a maximum number of ultrasound transducers 214 can be positioned on the guidewire 212. In another example, the one or more ultrasound transducers 214 may be arranged in linear arrays with each array extending generally parallel to a longitudinal axis of the guidewire 212. When so provided, the arrays may be uniformly or eccentrically spaced about a circumference of the guidewire 212. In some examples, the one or more ultrasound transducers 214 may be provided as circumferentially extending arrays with each array extending about a circumference of the guidewire 212. In yet other embodiments, the one or more ultrasound transducers 214 may be arranged in a helical manner along the guidewire 212. For example, an ultrasound transducer 214 may be longitudinally and circumferentially spaced from each adjacent ultrasound transducer 214. These are just some examples. It can be appreciated that the one or more ultrasound transducers 214 may be positioned in any arrangement, as desired. In some embodiments, each ultrasound transducer 214 may function independently from other transducers or the ultrasound transducers 214 may be collectively operated to effectively function as a single ultrasound transducer.

Each of the one or more ultrasound transducers 214 may produce an ultrasound field 218 that includes a near field region and a far field region. In the near field region, dynamic acoustic pressures may be cyclically applied to the calcified lesion 204. As used herein, the near field region refers to a region in close proximity radially to a surface of the ultrasound transducer 214, for example, the region extending outward from the transducer surface to a radial distance less than or equal to a length of the ultrasound transducer 214, wherein the acoustic pressure waves transmitted by the ultrasound transducer 214 are unfocused and can be controlled to be substantially uniform upon the calcified lesion 204. The ultrasound transducers 214 may be configured to emit ultrasound waves in two opposing directions, or about 180° apart. In other examples, the ultrasound transducers 214 may be configured to emit ultrasound waves in a single direction. For example, the ultrasound sound transducers 214 may be configured to emit ultrasound waves in a direction radially outward from the guidewire 212 or in a direction generally parallel to the longitudinal axis of the guidewire 212. When two or more ultrasound transducers 214 are provided, the ultrasound transducers 214 may be arranged to emit ultrasound waves in differing radial and/or axial directions, although this is not required.

In some cases, for example, the ultrasound transducer 214 may be configured to impart a uniform or substantially uniform acoustic pressure to an end of the calcified lesion 204. As the lesion 204 breaks down, the guidewire 212 may be advanced to break up a length of the lesion 204. In some instances, ultrasound transducers 214 may be positioned to emit ultrasound waves in a direction generally parallel to the longitudinal axis of the guidewire 212 to create a path through the lesion 204 for the guidewire 212 and/or guide catheter 206. Additional ultrasound transducers 214 may be positioned along a length of the guidewire 212 to emit radially extending ultrasound waves to further break down the lesion 204 as the guidewire 212 is passed therethrough. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter. Depending on the therapeutic applications, the ultrasound transducer 214 may be configured to impart a uniform or substantially uniform acoustic pressure over a length of about 10 mm to about 60 mm at a radial distance of about 1 mm to about 8 mm as measured from a central axis extending through the catheter shaft 18. Multiple ultrasound transducers 214 may be used and configured to extend the effective therapeutic length, such as up to a length of about 200 mm.

To impart a uniform or substantially uniform acoustic pressure in the near field, the ultrasound transducers 214 may have a length that is multiple times larger than a diameter of the guidewire 212. In some cases, the ultrasound transducers 214 may have a length that is at least as long as a length of the calcified lesion 204, to generate a uniform or substantially uniform acoustic pressure over a length of about 20 to about 80 mm. As described above, the ultrasound transducers 214, may be a single ultrasound transducer or a series of ultrasound transducers or transducer elements driven in such a way as to effectively act as a single ultrasound transducer. The ultrasound transducers 214 may be electrically coupled to an electronic source (not explicitly shown) via one or more wires (not explicitly shown). In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 204.

In use, the guidewire 212 may be advanced through the vasculature to a desired treatment region. Energy may then be supplied to the ultrasound transducer 214. The application of the sound energy may break up the plaque/lesion to allow the guidewire 212 and/or guide catheter 206 to pass the occlusion 204. In some cases, once the occlusion 204 has been broken up, the guidewire 212 may be longitudinally advanced. While not explicitly shown, the ultrasound transducers 214 may be connected to a single control unit or to separate control units by one or more electrical conductors. The amount of energy delivered to the ultrasound transducers may be determined by the desired treatment as well as the feedback provided by other components of the catheter or other devices.

Once a particular location has been treated, it may be desirable to perform further procedures at different longitudinal locations. The guidewire 212 may be longitudinally repositioned and energy may once again be delivered to the ultrasound transducer 214. If necessary, the guidewire 212 may be rotated to break up the lesion around the circumference of the vessel at each longitudinal location. This process may be repeated at any number of longitudinal locations desired.

In some embodiments, an expandable metal or polymer frame may be used in addition to or in place of an inflatable balloon to anchor and apply force on the lesion while emitting sound energy to the target tissue. The expandable frame or basket may be configured to apply a targeted outward force on the lesion while using sound energy to break up the lesion. It is contemplated that the sound energy may be transmitted through blood and/or soft tissue to break up the lesion. In some cases, the use of an expandable basket may allow for blood flow to be maintained during the treatment.

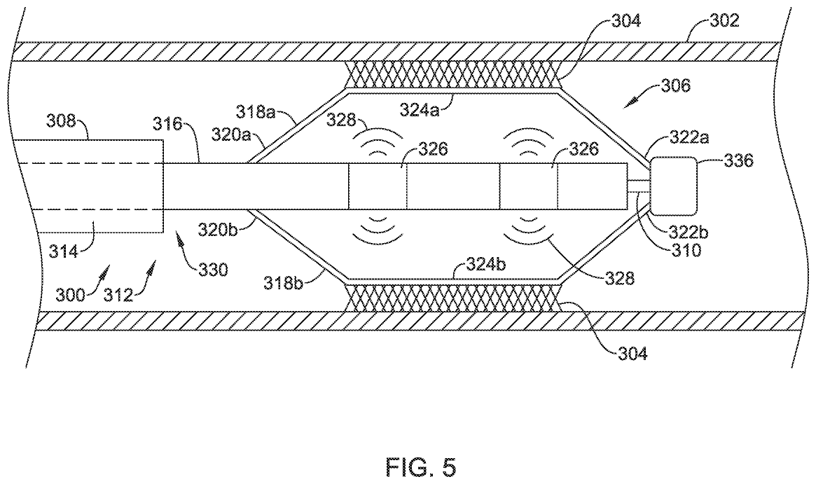

FIG. 5 illustrates a side view of another illustrative catheter 300 that may be used to treat a lesion disposed in a blood vessel 302 and positioned adjacent to an intravascular lesion 304. The catheter 300 may be configured to apply a force to the lesion 304 as well as emit a shockwave or ultrasound field.

The catheter 300 may include an expandable basket 306 coupled to a catheter shaft 308. The shaft 308 may be an outer tubular member or a catheter shaft, similar to typical catheter shafts. For example, the catheter shaft 308 may be a tubular member extending from a distal end region 312 to a proximal end region (not explicitly shown) configured to remain outside of a patient's body. A lumen 314 may extend from the distal end region 312 to the proximal end region.

The catheter 300 may further include an inner tubular member 316, which may be slidably disposed within the lumen 314 of the catheter shaft 308. At least a portion of the expandable basket 306 may be coupled to a distal end region 330 of the inner tubular member 316. The catheter shaft 308 and inner tubular member 316 may be manufactured from a number of different materials. For example, the catheter shaft 308 and inner tubular member 316 may be made of metals, metal alloys, polymers, metal-polymer composites or any other suitable materials.

The expandable basket 306 may be configured to transition between a collapsed configuration and an expanded configuration (FIG. 5). The expandable basket 306 may include a number of expandable positioning elements such as longitudinally extending struts 318a, 318b (collectively 318), which may be coupled to the inner tubular member 316 at their proximal ends 320a, 320b (collectively, 320). The distal ends 322a, 322b of the struts 318 may be coupled to a cap 336. In some instances, the cap 336 may include spacers which be used to maintain a consistent spacing between each of the struts 318. However, this is not required. In some cases, the struts 318 may be eccentrically arranged. The struts 318 may be configured to extend generally along the longitudinal axis of the catheter shaft 308. While the expandable basket 306 is illustrated as including two longitudinally extending struts 318, the expandable basket 306 may include any number of struts 318 desired, such as, but not limited to one, two, three, four, five, six, or more. Other suitable expandable positioning elements such as, but not limited to, rods or bars, a single hypotube having portions removed to form struts, an expandable stent (e.g., woven, braided, laser cut, etc.) having the proximal end and/or distal end gathered together, or the like may also be utilized.

The expandable basket 306 may be self-expandable or may require external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 306 may be heat set in the expanded state and then compressed to fit within the catheter shaft 308, for example. In another embodiment, a spring may be provided to effect expansion. Alternatively, external forces such as, but not limited to, pneumatic methods, compressed fluid, pull wires, push wires, or the like may also be employed to expand the expandable basket 306. It is contemplated that nickel-titanium alloys may enable kink-resistant folding and self-expansion. In other examples, magnetic alloys, metals, metal alloys, polymers, composites, etc. may be used to form the expandable basket 306.

In other instances, a manual force applied to the inner tubular member 316 may manipulate or actuate the expandable basket 306 between the expanded and collapsed state. For example, actuation element may include a central wire 310 that extends through the expandable basket 306 and is coupled to the cap 336. According to this embodiment, a pulling force exerted proximally on the wire may allow the struts 318 to expand and move the expandable basket 306 into an expanded state. A pushing force exerted distally on the wire may move elongate the struts 318 and/or otherwise shift the expandable basket 306 to a compressed or elongated state. Other actuation mechanisms may also be utilized.

As discussed above, the expandable basket 306 may include a number of expandable positioning elements such as longitudinally extending struts 318. The struts may each extend from a proximal end region 320 to a distal end region 322. An intermediate region 324a, 324b (collectively, 324) may be disposed between the proximal end regions 320 and the distal end regions 322. It is contemplated that in the expanded state, the intermediate regions 324 of the struts 318 may contact the vessel wall 302 (and/or lesion 304).

The inner tubular member 316 may include one or more ultrasound transducers or emitters 326. The ultrasound transducers 326 may be coupled to an outer surface of the inner tubular member 316 adjacent a distal portion thereof. During a procedure, the inner tubular member 316 may be positioned such that the ultrasound transducers 326 generally align with the lesion 304. In some cases, the ultrasound transducers 326 may include a piezoelectric material, which transmits acoustic pressure in response to an applied voltage. The ultrasound transducers 326 may be driven at one or more frequencies in the range of about 20 kilohertz (kHz) to about 50 megahertz (MHz). The ultrasound transducers 326 may be a single ultrasound transducer, or the ultrasound transducers 326 may include a series of ultrasound transducers that may be operated to effectively function as a single ultrasound transducer, providing the desired acoustic pressure over the desired treatment area. The acoustic pressure applied may range from tens of kiloPascals (kPa) to in excess of ten megaPascals (MPa).

The ultrasound transducers 326 may produce an ultrasound field 328 that includes a near field region and a far field region. In the near field region, dynamic acoustic pressures may be cyclically applied to the calcified lesion 304. As used herein, the near field region refers to a region in close proximity radially to a surface of the ultrasound transducers 326, for example, the region extending outward from the transducer surface to a radial distance less than or equal to a length of the ultrasound transducers 326, wherein the acoustic pressure waves transmitted by the ultrasound transducers 326 are unfocused and can be controlled to be substantially uniform upon the calcified lesion 304. The ultrasound transducers 326 may be configured to emit ultrasound waves in two opposing directions, or about 180° apart.

In some cases, for example, the ultrasound transducers 326 may be configured to impart a uniform or substantially uniform acoustic pressure along the length of the calcified lesion 304. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter. Depending on the therapeutic applications, the ultrasound transducers 326 may be configured to impart a uniform or substantially uniform acoustic pressure over a length of about 10 mm to about 60 mm at a radial distance of about 1 mm to about 8 mm as measured from a central axis extending through the catheter shaft 308. While not explicitly shown, multiple ultrasound transducers 326 may be used and configured to extend the effective therapeutic length, such as up to a length of about 200 mm.

To impart a uniform or substantially uniform acoustic pressure in the near field, the ultrasound transducers 326 may have a length that is multiple times larger than a diameter of the inner tubular member 316 and/or the catheter shaft 308. In some cases, the ultrasound transducer 326 may have a length that is at least as long as a length of the calcified lesion 304, to generate a uniform or substantially uniform acoustic pressure over a length of about to about 80 mm. In some instances, the ultrasound transducer 326, may be a single ultrasound transducer or a series of ultrasound transducers or transducer elements driven in such a way as to effectively act as a single ultrasound transducer. While not explicitly shown, the ultrasound transducer 326 may be electrically coupled to an electronic source via one or more wires. In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 304.

In use, the catheter 300 may be advanced through the vasculature to a desired treatment region. Once the catheter 300 is at the treatment region, the expandable basket 306 may then be expanded. Energy may then be supplied to the ultrasound transducers 326. The expansion of the expandable basket 306 and/or the application of the sound energy may break up the lesion 304 to dilate or expand the vessel 302. While not explicitly shown, the ultrasound transducers 326 may be connected to a single control unit or to separate control units by one or more electrical conductors (not explicitly shown). The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 300 may be longitudinally repositioned and energy may once again be delivered to the ultrasound transducers 326. If necessary, the catheter 300 may be rotated to break up the lesion around the circumference of the vessel at each longitudinal location. This process may be repeated at any number of longitudinal locations desired. It is contemplated that the expandable basket 306 may be collapsed or partially collapsed to allow for movement of the catheter 300. When the procedure has been completed, the expandable basket 306 may be collapsed for withdrawal from the body.

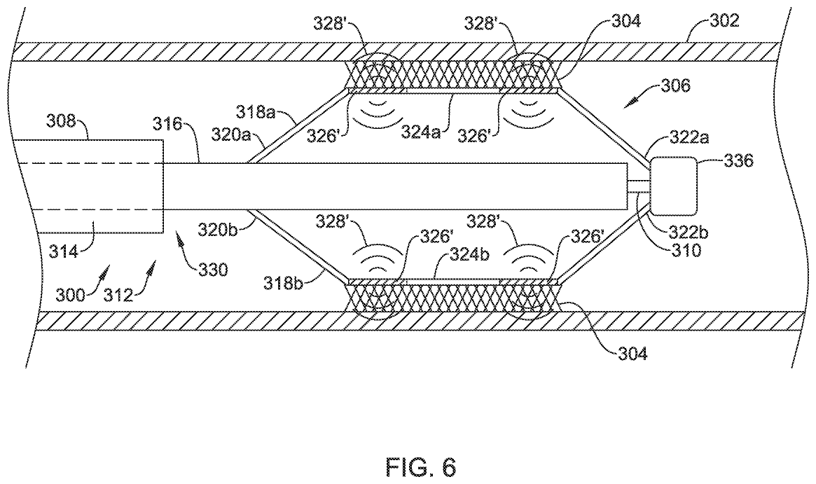

It is contemplated that the ultrasound transducers 326 may be positioned at locations in addition to or in place of the inner tubular member 316. FIG. 6 illustrates a side view of another illustrative catheter 300′ with one or more ultrasound transducers 326′ positioned on the expandable basket 306. The catheter 300′ may be similar in form and function to the catheter 300 described with respect to FIG. 5 where the same or similar reference numbers will be used to refer to the same or like parts. The one or more ultrasound transducers 326′ may be positioned on one or more of the longitudinally extending struts 318 of the expandable basket 306. In some examples, the one or more ultrasound transducers 326′ may be positioned on the intermediate portion 324 of the struts configured to contact the vessel wall 302, although this is not required. The one or more ultrasound transducers 326′ may be positioned along any portion of the struts 318 desired. It is contemplated that locating the ultrasound transducers 326 on the struts 318 may bring the ultrasound transducers in closer proximity to the lesion 304 for a targeted and/or localized therapy. The ultrasound waves 328′ may be transmitted through the vessel wall 302.

The catheter 300′ may include fewer than four ultrasound transducers 326′ or more than four ultrasound transducers 326′, as desired. It is further contemplated that additional ultrasound transducers 326′ may be provided on the inner tubular member 316, as shown in FIG. 15. When more than one ultrasound transducer 326′ is provided, the ultrasound transducers 326′ may be configured to be individually or collectively activated, as desired. While not explicitly shown, an electrical connector may extend from the ultrasound transducer 326′ to a control unit configured to remain outside the body. The electrical conductor may be disposed within a lumen 314 of the catheter shaft 308 or along an exterior of the catheter 300′, as desired.

In use, the catheter 300′ may be advanced through the vasculature to a desired treatment region. Once the catheter 300′ is at the treatment region, the expandable basket 306 may then be expanded to anchor the catheter 300′. Energy may then be supplied to the ultrasound transducer 326′. The application of the sound energy and the application of force via the expandable basket 306 may break up the plaque/lesion. While not explicitly shown, the ultrasound transducers 326′ may be connected to a single control unit or to separate control units by one or more electrical conductors. The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 300′ may be longitudinally repositioned and energy may once again be delivered to the ultrasound transducers 326′. If necessary, the catheter 300′ may be rotated to break up the lesions around the circumference of the vessel 302 at each longitudinal location. This process may be repeated at any number of longitudinal locations desired. It is contemplated that the expandable basket 306 may be collapsed to allow for movement of the catheter 300′. When the procedure has been completed, the expandable basket 306 may be collapsed for withdrawal from the body.

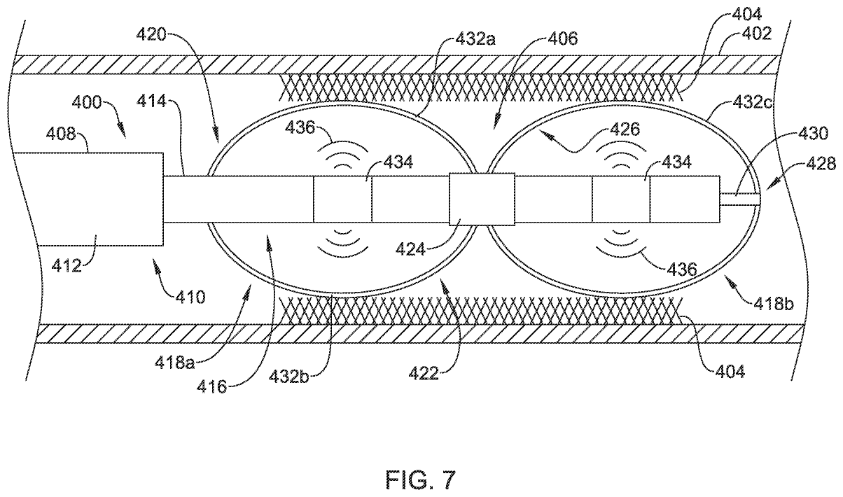

FIG. 7 illustrates a side view of another illustrative catheter 400 that may be used to treat a lesion disposed in a blood vessel 402 and positioned adjacent to an intravascular lesion 404. The catheter 400 may be configured to apply a force to the lesion 404 as well as emit a shockwave or ultrasound field.

The catheter 400 may include an expandable basket 406 coupled to a catheter shaft 408. The shaft 408 may be an outer tubular member or a catheter shaft, similar to typical catheter shafts. For example, the catheter shaft 408 may be a tubular member extending from a distal end region 410 to a proximal end region (not explicitly shown) configured to remain outside of a patient's body. A lumen 412 may extend from the distal end region 410 to the proximal end region.

The catheter 400 may further include an inner tubular member 414, which may be slidably disposed within the lumen 412 of the catheter shaft 408. At least a portion of the expandable basket 406 may be coupled to a distal end region 416 of the inner tubular member 414. The catheter shaft 408 and inner tubular member 414 may be manufactured from a number of different materials. For example, the catheter shaft 408 and inner tubular member 414 may be made of metals, metal alloys, polymers, metal-polymer composites or any other suitable materials.

The expandable basket 406 may be configured to transition between a collapsed configuration and an expanded configuration (FIG. 7). The expandable basket 406 may include a two or more expanding lobes 418a, 418b (collectively, 418). The proximal end 420 of the proximal lobe 418a may be coupled to the inner tubular member 414 while the distal end 422 of the proximal lobe 418a may be coupled to a slide ring 424. The proximal end 426 of the distal lobe 418b may be coupled to the slide ring 424 while the distal end 428 may be coupled to a pull wire 430. The slide ring 424 may couple the lobes 418a, 418b such that movement of one results in movement of the other. While the expandable basket 406 is illustrated as having two lobes 418, it is contemplated that the expandable basket 406 may include more than two lobes 418, as desired. Additional slide rings may be used to couple additional lobes, if so provided.

Each lobe 418 may include a number of expandable positioning elements such as longitudinally extending struts 432a, 432b, 432c, 432d (collectively 432). In some instances, the struts 432 of each lobe 418 may be uniformly spaced. However, this is not required. In some cases, the struts 432 of each lobe 418 may be eccentrically arranged. The struts 432 may be configured to extend generally along the longitudinal axis of the catheter shaft 408. While each lobe 418 is illustrated as including two longitudinally extending struts 432, each lobe 418 may include any number of struts 432 desired, such as, but not limited to one, two, three, four, five, six, or more. Further, each lobe 418 need not have the same number of struts 432 or have the same arrangement of struts 432. Other suitable expandable positioning elements such as, but not limited to, rods or bars, a single hypotube having portions removed to form struts, an expandable stent (e.g., woven, braided, laser cut, etc.) having the proximal end and/or distal end gathered together, or the like may also be utilized.

The expandable basket 406 may be self-expandable or may require external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 406 may be heat set in the expanded state and then compressed to fit within the catheter shaft 408, for example. In another embodiment, a spring may be provided to effect expansion. Alternatively, external forces such as, but not limited to, pneumatic methods, compressed fluid, pull wires, push wires, or the like may also be employed to expand the expandable basket 406. It is contemplated that nickel-titanium alloys may enable kink-resistant folding and self-expansion. In other examples, magnetic alloys, metals, metal alloys, polymers, composites, etc. may be used to form the expandable basket 406.

In other instances, a manual force applied to the inner tubular member 414 may manipulate or actuate the expandable basket 406 between the expanded and collapsed state. For example, an actuation element may include a central wire 430 that extends through the expandable basket 406 and is coupled to the distal end 428 of the distal lobe 418. According to this embodiment, a pulling force exerted proximally on the wire may allow the struts 432 to expand and move the expandable basket 406 into an expanded state. A pushing force exerted distally on the wire may move elongate the struts 432 and/or otherwise shift the ablation device to a compressed or elongated state. Other actuation mechanisms may also be utilized. For example, in some cases, the inner tubular member 414 may be actuated relative to the central wire 430.

As discussed above, the expandable basket 406 may include a number of expandable positioning elements such as longitudinally extending struts 432. The struts may each extend from a proximal end region to a distal end region. An intermediate region may be disposed between the proximal end regions and the distal end regions. It is contemplated that in the expanded state, the intermediate regions of the struts 432 may contact the vessel wall 402 (and/or lesion 404).

The inner tubular member 414 may include one or more ultrasound transducers or emitters 434. The ultrasound transducers 434 may be coupled to an outer surface of the inner tubular member 414 adjacent a distal portion thereof. Alternatively, or additionally, one or more ultrasound transducers may be positioned on one or more of the struts 432. During a procedure, the inner tubular member 414 may be positioned such that the ultrasound transducers 434 generally align with the lesion 404. In some cases, the ultrasound transducers 434 may include a piezoelectric material, which transmits acoustic pressure in response to an applied voltage. The ultrasound transducers 434 may be driven at one or more frequencies in the range of about 20 kilohertz (kHz) to about 50 megahertz (MHz). The ultrasound transducers 434 may be a single ultrasound transducer, or the ultrasound transducers 434 may include a series of ultrasound transducers that may be operated to effectively function as a single ultrasound transducer, providing the desired acoustic pressure over the desired treatment area. The acoustic pressure applied may range from tens of kiloPascals (kPa) to in excess of ten megaPascals (MPa).

The ultrasound transducers 434 may produce an ultrasound field 436 that includes a near field region and a far field region. In the near field region, dynamic acoustic pressures may be cyclically applied to the calcified lesion 404. As used herein, the near field region refers to a region in close proximity radially to a surface of the ultrasound transducers 434, for example, the region extending outward from the transducer surface to a radial distance less than or equal to a length of the ultrasound transducers 434, wherein the acoustic pressure waves transmitted by the ultrasound transducers 434 are unfocused and can be controlled to be substantially uniform upon the calcified lesion 404. The ultrasound transducers 434 may be configured to emit ultrasound waves in two opposing directions, or about 180° apart. When two or more ultrasound transducers 434 are provided, the ultrasound transducers 434 may be arranged to emit ultrasound waves in differing radial directions, although this is not required.

In some cases, for example, the ultrasound transducers 434 may be configured to impart a uniform or substantially uniform acoustic pressure along the length of the calcified lesion 404. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter. Depending on the therapeutic applications, the ultrasound transducers 434 may be configured to impart a uniform or substantially uniform acoustic pressure over a length of about 10 mm to about 60 mm at a radial distance of about 1 mm to about 8 mm as measured from a central axis extending through the catheter shaft 408. While not explicitly shown, multiple ultrasound transducers 434 may be used and configured to extend the effective therapeutic length, such as up to a length of about 200 mm.

To impart a uniform or substantially uniform acoustic pressure in the near field, the ultrasound transducers 434 may have a length that is multiple times larger than a diameter of the inner tubular member 414 and/or the catheter shaft 408. In some cases, the ultrasound transducer 434 may have a length that is at least as long as a length of the calcified lesion 404, to generate a uniform or substantially uniform acoustic pressure over a length of about to about 80 mm. In some instances, the ultrasound transducer 434, may be a single ultrasound transducer or a series of ultrasound transducers or transducer elements driven in such a way as to effectively act as a single ultrasound transducer. While not explicitly shown, the ultrasound transducer 434 may be electrically coupled to an electronic source via one or more wires. In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 404.

In use, the catheter 400 may be advanced through the vasculature to a desired treatment region. Once the catheter 400 is at the treatment region, the expandable basket 406 may then be expanded. Energy may then be supplied to the ultrasound transducers 434. The expansion of the expandable basket 406 and/or the application of the sound energy may break up the lesion 404 to dilate or expand the vessel 402. While not explicitly shown, the ultrasound transducers 434 may be connected to a single control unit or to separate control units by one or more electrical conductors (not explicitly shown). The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 400 may be longitudinally repositioned and energy may once again be delivered to the ultrasound transducers 434. If necessary, the catheter 400 may be rotated to perform ablation around the circumference of the vessel at each longitudinal location. This process may be repeated at any number of longitudinal locations desired. It is contemplated that the expandable basket 406 may be collapsed or partially collapsed to allow for movement of the catheter 400. When the procedure has been completed, the expandable basket 406 may be collapsed for withdrawal from the body.

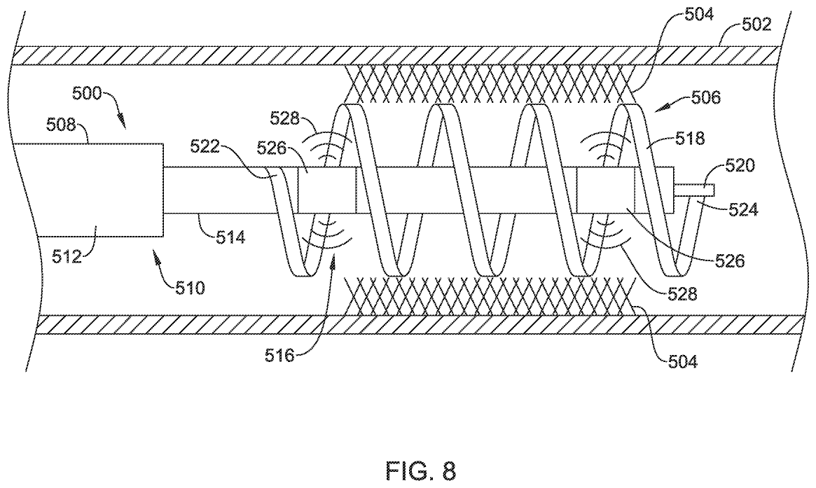

FIG. 8 illustrates a side view of another illustrative catheter 500 that may be used to treat a lesion disposed in a blood vessel 502 and positioned adjacent to an intravascular lesion 504. The catheter 500 may be configured to apply a force to the lesion 504 as well as emit a shockwave or ultrasound field.

The catheter 500 may include an expandable basket 506 coupled to a catheter shaft 508. The shaft 508 may be an outer tubular member or a catheter shaft, similar to typical catheter shafts. For example, the catheter shaft 508 may be a tubular member extending from a distal end region 510 to a proximal end region (not explicitly shown) configured to remain outside of a patient's body. A lumen 512 may extend from the distal end region 510 to the proximal end region.

The catheter 500 may further include an inner tubular member 514, which may be slidably disposed within the lumen 512 of the catheter shaft 508. At least a portion of the expandable basket 506 may be coupled to a distal end region 516 of the inner tubular member 514. The catheter shaft 508 and inner tubular member 514 may be manufactured from a number of different materials. For example, the catheter shaft 508 and inner tubular member 514 may be made of metals, metal alloys, polymers, metal-polymer composites or any other suitable materials.

The expandable basket 506 may be configured to transition between a collapsed configuration and an expanded configuration (FIG. 8). The expandable basket 506 may include a generally helically extending strut 518. A proximal end 522 of the strut 518 may be coupled to the inner tubular member 514 while a distal end 524 of the strut 518 may be coupled to a central wire 520. The strut 518 may generally take the shape of a spring. It is contemplated that the distance between adjacent windings may be increased or decreased, as desired. In some instances, the distance between adjacent windings may be uniformly spaced. However, this is not required. In some cases, the distance between adjacent windings may be eccentrically arranged. The strut 518 may be configured to extend along a length of the inner tubular member 514 of the catheter shaft 508. While the expandable basket 506 is illustrated as including one strut 518, the expandable basket 506 may include any number of struts 518 desired, such as, but not limited to one, two, three, four, five, six, or more. Other suitable expandable positioning elements such as, but not limited to, rods or bars, a single hypotube having portions removed to form struts, an expandable stent (e.g., woven, braided, laser cut, etc.) having the proximal end and/or distal end gathered together, or the like may also be utilized.

The expandable basket 506 may be self-expandable or may require external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 506 may be heat set in the expanded state and then compressed to fit within the catheter shaft 508, for example. In another embodiment, a spring may be provided to effect expansion. Alternatively, external forces such as, but not limited to, pneumatic methods, compressed fluid, pull wires, push wires, or the like may also be employed to expand the expandable basket 506. It is contemplated that nickel-titanium alloys may enable kink-resistant folding and self-expansion. In other examples, magnetic alloys, metals, metal alloys, polymers, composites, etc. may be used to form the expandable basket 506.

In other instances, a manual force applied to the inner tubular member 514 may manipulate or actuate the expandable basket 506 between the expanded and collapsed state. For example, an actuation element may include a central wire 520 that extends through the expandable basket 506 and is coupled to a distal end 524 of the strut 518. According to this embodiment, a pulling force exerted proximally on the wire may allow the strut 518 to expand and move the expandable basket 506 into an expanded state. A pushing force exerted distally on the wire may move elongate the strut 518 and/or otherwise shift the ablation device to a compressed or elongated state. Other actuation mechanisms may also be utilized. For example, in some cases, the inner tubular member 514 may be actuated relative to the central wire 520.

The inner tubular member 514 may include one or more ultrasound transducers or emitters 526. The ultrasound transducers 526 may be coupled to an outer surface of the inner tubular member 514 adjacent a distal portion thereof. Alternatively, or additionally, one or more ultrasound transducers may be positioned on the strut 518. During a procedure, the inner tubular member 514 may be positioned such that the ultrasound transducers 526 generally align with the lesion 504. In some cases, the ultrasound transducers 526 may include a piezoelectric material, which transmits acoustic pressure in response to an applied voltage. The ultrasound transducers 526 may be driven at one or more frequencies in the range of about 20 kilohertz (kHz) to about 50 megahertz (MHz). The ultrasound transducers 526 may be a single ultrasound transducer, or the ultrasound transducers 526 may include a series of ultrasound transducers that may be operated to effectively function as a single ultrasound transducer, providing the desired acoustic pressure over the desired treatment area. The acoustic pressure applied may range from tens of kiloPascals (kPa) to in excess of ten megaPascals (MPa).

The ultrasound transducers 526 may produce an ultrasound field 528 that includes a near field region and a far field region. In the near field region, dynamic acoustic pressures may be cyclically applied to the calcified lesion 504. As used herein, the near field region refers to a region in close proximity radially to a surface of the ultrasound transducers 526, for example, the region extending outward from the transducer surface to a radial distance less than or equal to a length of the ultrasound transducers 526, wherein the acoustic pressure waves transmitted by the ultrasound transducers 526 are unfocused and can be controlled to be substantially uniform upon the calcified lesion 504. The ultrasound transducers 526 may be configured to emit ultrasound waves in two opposing directions, or about 180° apart. When two or more ultrasound transducers 526 are provided, the ultrasound transducers 526 may be arranged to emit ultrasound waves in differing radial directions, although this is not required.

In some cases, for example, the ultrasound transducers 526 may be configured to impart a uniform or substantially uniform acoustic pressure along the length of the calcified lesion 504. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter. Depending on the therapeutic applications, the ultrasound transducers 526 may be configured to impart a uniform or substantially uniform acoustic pressure over a length of about 10 mm to about 60 mm at a radial distance of about 1 mm to about 8 mm as measured from a central axis extending through the catheter shaft 508. While not explicitly shown, multiple ultrasound transducers 526 may be used and configured to extend the effective therapeutic length, such as up to a length of about 200 mm.

To impart a uniform or substantially uniform acoustic pressure in the near field, the ultrasound transducers 526 may have a length that is multiple times larger than a diameter of the inner tubular member 514 and/or the catheter shaft 508. In some cases, the ultrasound transducer 526 may have a length that is at least as long as a length of the calcified lesion 504, to generate a uniform or substantially uniform acoustic pressure over a length of about 20 to about 80 mm. In some instances, the ultrasound transducer 526, may be a single ultrasound transducer or a series of ultrasound transducers or transducer elements driven in such a way as to effectively act as a single ultrasound transducer. While not explicitly shown, the ultrasound transducer 526 may be electrically coupled to an electronic source via one or more wires. In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 504.

In use, the catheter 500 may be advanced through the vasculature to a desired treatment region. Once the catheter 500 is at the treatment region, the expandable basket 506 may then be expanded. Energy may then be supplied to the ultrasound transducers 526. The expansion of the expandable basket 506 and/or the application of the sound energy may break up the lesion 504 to dilate or expand the vessel 502. While not explicitly shown, the ultrasound transducers 526 may be connected to a single control unit or to separate control units by one or more electrical conductors (not explicitly shown). The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 500 may be longitudinally repositioned and energy may once again be delivered to the ultrasound transducers 526. If necessary, the catheter 500 may be rotated to perform ablation around the circumference of the vessel at each longitudinal location. This process may be repeated at any number of longitudinal locations desired. It is contemplated that the expandable basket 506 may be collapsed or partially collapsed to allow for movement of the catheter 500. When the procedure has been completed, the expandable basket 506 may be collapsed for withdrawal from the body.

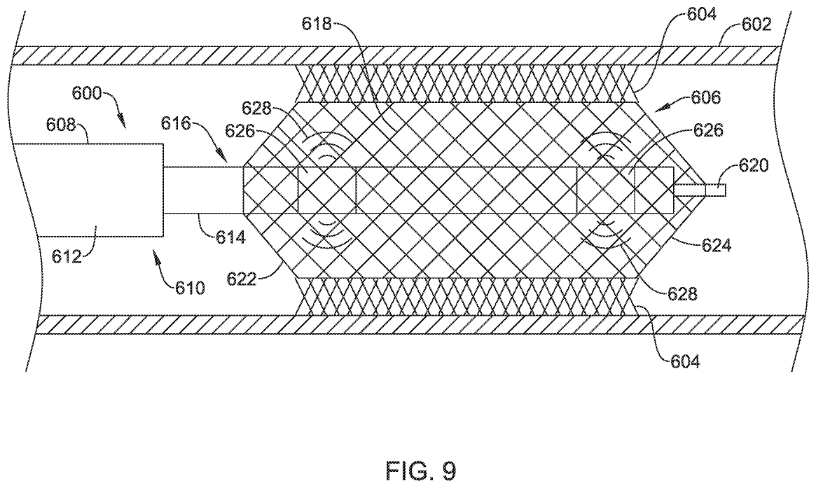

FIG. 9 illustrates a side view of another illustrative catheter 600 that may be used to treat a lesion disposed in a blood vessel 602 and positioned adjacent to an intravascular lesion 604. The catheter 600 may be configured to apply a force to the lesion 604 as well as emit a shockwave or ultrasound field.

The catheter 600 may include an expandable basket 606 coupled to a catheter shaft 608. The shaft 608 may be an outer tubular member or a catheter shaft, similar to typical catheter shafts. For example, the catheter shaft 608 may be a tubular member extending from a distal end region 610 to a proximal end region (not explicitly shown) configured to remain outside of a patient's body. A lumen 612 may extend from the distal end region 610 to the proximal end region.

The catheter 600 may further include an inner tubular member 614, which may be slidably disposed within the lumen 612 of the catheter shaft 608. At least a portion of the expandable basket 606 may be coupled to a distal end region 616 of the inner tubular member 614. The catheter shaft 608 and inner tubular member 614 may be manufactured from a number of different materials. For example, the catheter shaft 608 and inner tubular member 614 may be made of metals, metal alloys, polymers, metal-polymer composites or any other suitable materials.

The expandable basket 606 may be configured to transition between a collapsed configuration and an expanded configuration (FIG. 9). A proximal end 622 of the expandable basket 606 may be coupled to the inner tubular member 614 while a distal end 624 of the expandable basket 606 may be coupled to a central wire 620. The expandable basket 606 may have a woven structure, fabricated from a number of filaments or struts 618 forming a generally tubular wall. In some embodiments, the expandable basket 606 may be knitted or braided with a single filament or strut interwoven with itself and defining open cells extending through the thickness of the tubular wall of the expandable basket 606. In other embodiments, the expandable basket 606 may be braided with several filaments or struts interwoven together and defining open cells extending along a length and around the circumference of the tubular wall of the expandable basket 606. In another embodiment, the expandable basket 606 may be knitted. In yet another embodiment, the expandable basket 606 may be of a knotted type. In still another embodiment, the expandable basket 606 may be a laser cut tubular member. A laser cut tubular member may have an open and/or closed cell geometry including one or more interconnected monolithic filaments or struts defining open cells therebetween, with the open cells extending along a length and around the circumference of the tubular wall.

The expandable basket 606 may be self-expandable or may require external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 606 may be heat set in the expanded state and then compressed to fit within the catheter shaft 608, for example. In another embodiment, a spring may be provided to effect expansion. Alternatively, external forces such as, but not limited to, pneumatic methods, compressed fluid, pull wires, push wires, or the like may also be employed to expand the expandable basket 606. It is contemplated that nickel-titanium alloys may enable kink-resistant folding and self-expansion. In other examples, magnetic alloys, metals, metal alloys, polymers, composites, etc. may be used to form the expandable basket 606.

In other instances, a manual force applied to the inner tubular member 614 may manipulate or actuate the expandable basket 606 between the expanded and collapsed state. For example, an actuation element may include a central wire 620 that extends through the expandable basket 606 and is coupled to a distal end 624 of the expandable basket 606. According to this embodiment, a pulling force exerted proximally on the wire may allow the expandable basket 606 to expand and move the expandable basket 606 into an expanded state. A pushing force exerted distally on the wire may move elongate the expandable basket 606 and/or otherwise shift the ablation device to a compressed or elongated state. Other actuation mechanisms may also be utilized. For example, in some cases, the inner tubular member 614 may be actuated relative to the central wire 620.

The inner tubular member 614 may include one or more ultrasound transducers or emitters 626. The ultrasound transducers 626 may be coupled to an outer surface of the inner tubular member 614 adjacent a distal portion thereof. Alternatively, or additionally, one or more ultrasound transducers may be positioned on the expandable basket 606. During a procedure, the inner tubular member 614 may be positioned such that the ultrasound transducers 626 generally align with the lesion 604. In some cases, the ultrasound transducers 626 may include a piezoelectric material, which transmits acoustic pressure in response to an applied voltage. The ultrasound transducers 626 may be driven at one or more frequencies in the range of about 20 kilohertz (kHz) to about 50 megahertz (MHz). The ultrasound transducers 626 may be a single ultrasound transducer, or the ultrasound transducers 626 may include a series of ultrasound transducers that may be operated to effectively function as a single ultrasound transducer, providing the desired acoustic pressure over the desired treatment area. The acoustic pressure applied may range from tens of kiloPascals (kPa) to in excess of ten megaPascals (MPa).

The ultrasound transducers 626 may produce an ultrasound field 628 that includes a near field region and a far field region. In the near field region, dynamic acoustic pressures may be cyclically applied to the calcified lesion 604. As used herein, the near field region refers to a region in close proximity radially to a surface of the ultrasound transducers 626, for example, the region extending outward from the transducer surface to a radial distance less than or equal to a length of the ultrasound transducers 626, wherein the acoustic pressure waves transmitted by the ultrasound transducers 626 are unfocused and can be controlled to be substantially uniform upon the calcified lesion 604. The ultrasound transducers 626 may be configured to emit ultrasound waves in two opposing directions, or about 180° apart. When two or more ultrasound transducers 626 are provided, the ultrasound transducers 626 may be arranged to emit ultrasound waves in differing radial directions, although this is not required.

In some cases, for example, the ultrasound transducers 626 may be configured to impart a uniform or substantially uniform acoustic pressure along the length of the calcified lesion 604. In cardiac vessel disease states, vascular lesions may span a length of about 10 millimeters (mm) to about 25 mm in vessels that are about 2 mm to about 4 mm in diameter. In peripheral vessel disease states, vascular lesions may span a length of up to about 200 mm in vessels up to about 12 mm in diameter. Depending on the therapeutic applications, the ultrasound transducers 626 may be configured to impart a uniform or substantially uniform acoustic pressure over a length of about 10 mm to about 60 mm at a radial distance of about 1 mm to about 8 mm as measured from a central axis extending through the catheter shaft 608. While not explicitly shown, multiple ultrasound transducers 626 may be used and configured to extend the effective therapeutic length, such as up to a length of about 200 mm.

To impart a uniform or substantially uniform acoustic pressure in the near field, the ultrasound transducers 626 may have a length that is multiple times larger than a diameter of the inner tubular member 614 and/or the catheter shaft 608. In some cases, the ultrasound transducer 626 may have a length that is at least as long as a length of the calcified lesion 604, to generate a uniform or substantially uniform acoustic pressure over a length of about 20 to about 80 mm. In some instances, the ultrasound transducer 626, may be a single ultrasound transducer or a series of ultrasound transducers or transducer elements driven in such a way as to effectively act as a single ultrasound transducer. While not explicitly shown, the ultrasound transducer 626 may be electrically coupled to an electronic source via one or more wires. In some cases, two or more ultrasound transducers may be coupled to a single electronic source and driven with the same frequency and output. In other embodiments, two or more ultrasound transducers may be coupled to two or more differing electronic sources and driven independently of one another so that amplitude and phase control may be applied to increase the uniformity of the acoustic pressure imparted to the lesion 604.

In use, the catheter 600 may be advanced through the vasculature to a desired treatment region. Once the catheter 600 is at the treatment region, the expandable basket 606 may then be expanded. Energy may then be supplied to the ultrasound transducers 626. The expansion of the expandable basket 606 and/or the application of the sound energy may break up the lesion 604 to dilate or expand the vessel 602. While not explicitly shown, the ultrasound transducers 626 may be connected to a single control unit or to separate control units by one or more electrical conductors (not explicitly shown). The amount of energy delivered to the ultrasound transducers may be determined by the desired treatment as well as the feedback provided by other components of the catheter or other devices.

Once a particular location has been treated, it may be desirable to perform further procedures at different longitudinal locations. The catheter 600 may be longitudinally repositioned and energy may once again be delivered to the ultrasound transducers 626. If necessary, the catheter 600 may be rotated to perform ablation around the circumference of the vessel at each longitudinal location. This process may be repeated at any number of longitudinal locations desired. It is contemplated that the expandable basket 606 may be collapsed or partially collapsed to allow for movement of the catheter 600. When the procedure has been completed, the expandable basket 606 may be collapsed for withdrawal from the body.

The materials that can be used for the various components of the system(s) and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the catheter shaft, the inflatable balloon, the expandable basket, the central wire, etc., and/or elements or components thereof.

In some embodiments, the system, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), expanded polytetrafluoroethylene (ePTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments components can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system, and/or components or portions thereof, may be made of a material that does not substantially distort the image and create substantial artifacts (i.e., gaps in the image). Certain ferromagnetic materials, for example, may not be suitable because they may create artifacts in an MRI image. The system, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and me s alamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the present disclosure. This may include, to the extent that it is appropriate, the use of any of the features of one example embodiment being used in other embodiments. The scope of the present disclosure is, of course, defined in the language in which the appended claims are expressed.

What is claimed is:

1. A method for breaking down an intravascular lesion, the method comprising:
advancing a catheter through a vasculature system to a target location, the catheter comprising:
a catheter shaft;
an expandable member secured to a distal portion of the catheter shaft; and
one or more ultrasound transducers; and
activating the one or more ultrasound transducers to emit an ultrasound field, the ultrasound field directed towards the target location.

2. The method of claim 1, wherein at least one of the one or more ultrasound transducers are coupled to the expandable member.

3. The method of claim 1, wherein the catheter shaft comprises an outer tubular member and an inner tubular member and at least one of the one or more ultrasound transducers are coupled to the inner tubular member.

4. The method of claim 1, wherein the expandable member comprises an inflatable balloon.

5. The method of claim 1, wherein the expandable member comprises an expandable basket.

6. The method of claim 1, wherein the one or more ultrasound transducers are arranged in one or more arrays.

7. The method of claim 1, wherein the ultrasound field is emitted radially from the catheter.

8. The method of claim 1, wherein the ultrasound field is emitted in a direction parallel to a longitudinal axis of the catheter shaft.

9. A method for breaking down an intravascular lesion, the method comprising:
advancing a catheter through a vasculature system to a target location, the catheter comprising:
a catheter shaft comprising an outer tubular member and an inner tubular member;
an inflatable balloon secured to a distal portion of the catheter shaft; and
one or more ultrasound transducers; and
activating the one or more ultrasound transducers to emit an ultrasound field, the ultrasound field directed towards the target location.

10. The method of claim 9, wherein at least one of the one or more ultrasound transducers are coupled to the inflatable balloon.

11. The method of claim 9, wherein at least one of the one or more ultrasound transducers are coupled to the inner tubular member.

12. The method of claim 9, wherein the ultrasound field is emitted radially from the catheter.

13. The method of claim 9, wherein the ultrasound field is emitted in a direction parallel to a longitudinal axis of the catheter shaft.

14. A method for breaking down an intravascular lesion, the method comprising:
advancing a catheter through a vasculature system to a target location, the catheter comprising:
a catheter shaft comprising an outer tubular member and an inner tubular member;
an expandable basket secured to a distal portion of the catheter shaft; and
one or more ultrasound transducers; and
activating the one or more ultrasound transducers to emit an ultrasound field, the ultrasound field directed towards the target location.

15. The method of claim 14, wherein at least one of the one or more ultrasound transducers are coupled to the expandable basket.

16. The method of claim 14, wherein at least one of the one or more ultrasound transducers are coupled to the inner tubular member.

17. The method of claim 14, wherein the expandable basket comprises two or more lobes.

18. The method of claim 14, wherein the expandable basket comprises one or more longitudinally extending struts.

19. The method of claim 14, wherein the expandable basket comprises a helically extending strut.

20. The method of claim 14, wherein the expandable basket comprises a woven structure.
